(19) United States
(12) Patent Application Publication
(10) Pub. No.: US 2005/0208020 A1
(43) Pub. Date: Sep. 22, 2005

(54) Enhancement of vaccine-induced immune responses and protection by heterologous boosting with alphavirus replicon vaccines

(76) Inventors: Denise L. Doolan, Rockville, MD (US); Gary T. Brice, (US); Daniel J. Carucci, Washington, DC (US); Kurt Kamrud, Apex, NC (US); Jeffrey Chulay, Chapel Hill, NC (US); Jonathan F. Smith, Cary, NC (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 10/988,403

(22) Filed: Nov. 10, 2004

Related U.S. Application Data
(60) Provisional application No. 60/518,872, filed on Nov. 12, 2003.

(51) Int. Cl. A61K 48/00; A61K 39/12

(52) U.S. Cl. 424/93.2; 424/199.1

(57) ABSTRACT

The inventive subject matter relates to an immunogenic composition and method of enhancing immunogenicity and protective immunity induced by any subunit or whole organism vaccine or combination of vaccines comprising administering to the subject a priming immunization preparation containing an antigen or fragment thereof, said preparation being selected from the group consisting of: a recombinant virus expression system; a recombinant protein antigen or a recombinant polypeptide; a synthetic peptide; a polynucleotide vector; a whole organism or extract and combinations thereof; and a boosting immunization of at least one alphavirus replicon containing an antigen or fragment thereof. The inventive subject matter further relates to an immunogenic composition and method of inducing an immune response that activates both cellular and humoral arms of the immune system.

### FIELD OF THE INVENTION

The inventive subject matter relates to an immunogenic composition and method of enhancing and broadening the immunogenicity and protective immunity in humans and animals induced by any subunit or whole organism vaccine or combination of vaccines comprising immunizing with a priming immunization mixture that contains recombinant poxviruses; recombinant adenoviruses; recombinant proteins; synthetic peptides; plasmid DNA; or live, attenuated or killed organisms, or extracts thereof, and subsequently immunizing with a boosting immunization preparation of an alphavirus replicon vaccine or combination of alphavirus replicon vaccines.

### BACKGROUND OF THE INVENTION

Malaria poses an enormous burden on public health throughout the world. It is a public health problem in more than 90 countries, inhabited by a total of some 2.4 billion people or 40% of the world's population. Worldwide incidence of the disease is estimated to be on the order of 300-500 million new infections and 2-4 million deaths annually r. Mortality due to malaria is estimated to be in the range of 1.5 to 2.7 million deaths annually according to the World Health Organization (WHO). In addition, tens of millions of travelers from North America, Europe, Japan or Australia visit areas of the world with malaria every year. Of these, 10,000-30,000 contract malaria annually. Furthermore, in every military campaign of the past century mounted in areas where malaria was transmitted, U.S. forces have suffered more casualties to malaria than from hostile fire, and entire divisions have been rendered non-operative. Finally, in sub-Saharan Africa, it is estimated that annually 1%-4% of gross domestic product (GDP), a minimum of $12 billion, is lost due to malaria (Gallup and Sachs, 1998). The cost of physical intervention methods intended to interfere with the transmission of the disease such as bednets and window screens is often prohibitive and such measures are not highly effective, and the availability and cost of prophylactic drugs precludes their use by many of individuals who need them the most. Moreover, the emergence of drug-resistant parasites means that many of the prophylactic drugs that were effective in the past are no longer useful, and many of the newer generation drugs are associated with rare but significant side effects, such as fatal heart rhythms, fatal skin disease, neurological disturbances, or gastrointestinal distress. The increase in insecticide-resistance of the vectors that transmit malaria, and the undesirable environmental impact of those insecticides shown to be most effective means that chemical interventions are frequently not useful in combating the disease. These factors emphasize the urgent need for the development of an effective malaria vaccine. The current status of malaria vaccine development and clinical trials have been the subject of a number of recent reviews (Graves & Gelband 2003: Moore et al. 2002; Carvalho et al. 2002; Moorthy & Hill 2002; Greenwood & Alonso 2002; Richie & Saul 2002). Over the past 15-20 years a series of Phase 1/2 vaccine trials have been reported using synthetic peptides or recombinant proteins based on malarial antigens.

Approximately 40 trials were reported up to 1998 (Engers & Godal 1998). Most of these have been directed against sporozoites or liver stages where the use of experimental mosquito challenges allows rapid progress through Phase 1 to Phase 2a preliminary efficacy studies. Anti-sporozoite vaccines tested have included completely synthetic peptides, conjugates of synthetic peptide with proteins such as tetanus toxoid to provide T cell help, recombinant malaria proteins, particle-forming recombinant chimeric constructs, recombinant viruses and bacteria and DNA vaccines. Several trials of asexual blood stage vaccines have used either synthetic peptide conjugates or recombinant proteins and there has been a single trial of a transmission blocking vaccine (recombinant Pfs25). A recurring theme has been the difficulty of obtaining a sufficiently strong and long lasting immune response in humans even though the same vaccine preparation is often strongly immunogenic in test animals Various strategies seek to overcome this limitation including the exploration of potent immune-stimulatory conjugates or adjuvants to boost the human response, or the development and application of novel vaccine technology platforms and application either alone or in combination with other technologies. The former approach is best illustrated by vaccines directed against the circumsporozoite protein (CSP), the prinicpal sporozotie coat protein. Early studies with both recombinant proteins, peptide conjugates and recombinant protein conjugates were able to elicit anti-CSP antibodies, provided marginal protection in Phase 2a studies and no protection in field studies More recently, a chimeric protein consisting of a fusion between the CSP and the hepatitis B surface antigen which forms typical HBsAg particles when expressed in the presence of unmodified HBsAg (the RTS,S vaccine) has been extensively evaluated in animals and in clinical studies with experimental challenge and field exposure (Kester et al., 2001; Bojan et al., 2001). Although much more immunogenic than recombinant CS protein, the RTS,S is still suboptimal with regard to its ability to protect against malaria. For example, although the RTS,S/ASO2 vaccine could protects 40-50% of volunteers experimentally challenged 2-3 weeks after their last immunization (Stoute 1997, Stoute 1998, Kester 2001), when only one of five protected volunteers was protected when rechallenged 6 months after the last immunization (Stoute 1998), and in field studies vaccine efficacy (end-point defined as “time to first infection”) was 71% (95% Cl 46% to 85%) during the first 9 weeks of surveillance but subsequently declined to 0% (95% Cl-52% to 34%) in the last 6 weeks (Bojang 2001). Although considerable efforts are still being directed at the development of protein-based vaccines, alternative technologies such as DNA and viral based vaccines show some promise with regard to immunogenicity and protective efficacy, at least in animal models; additionally, these molecular based vaccines may prove particularly amenable to multivalent formulations and ultimately less expensive to produce, store and deliver as compared with the more conventional vaccines.

In the malaria model, we and others have established the capacity of DNA vaccines encoding Plasmodium antigens to induce CD8⁺ CTL and IFN-γ responses and protection against sporozoite challenge in mice (Sedegah 1994; Doolan 1996).

In the malaria model, we and others have established the capacity of DNA vaccines encoding Plasmodium antigens to induce CD8⁺ CTL and IFN-γ responses and protection against sporozoite challenge in mice (Sedegah 1994; Doolan 1996) and monkeys (Wang 1998a; Rogers 2001, 2002; W. R. Weiss, unpub;ished), and Phase 1 and 2a clinical trials have established the safety, tolerability and immunogenicity of DNA vaccines encoding malaria antigens in normal healthy humans (Wang 1998b, 2000; Le 1999, Epstein 2001, T. Richie, unpublished). However the immunogenicity of first and second-generation DNA vaccines in nonhuman primates and in humans has been suboptimal. Even in murine models, DNA vaccines are not effective at activating all arms of he immune system. For example, immunization of mice with plasmid DNA encoding the pre-erythrocytic stage Plasmodium yoelii antigens, PyCSP and PyHEP17 induces antigen-specific cell mediated immune responses and antibody responses and confers sterile protection against sporozoite challenge However, this protection does not withstand high challenge doses, is not sustained for a long period (M. Sedegah, unpublished; D. L. Doolan, unpublished), and is genetically restricted (Doolan, 1996). In the protected mouse strains, PyCSP DNA induces good CD8⁺ CTL responses, good antibody responses, but poor CD4⁺ T cell responses (Sedegah 1994, 1998; W. R. Weiss, unpublished), and PyHEP17 induces poor CD8⁺ CTL and CD4⁺ T cell responses and negligible antibody responses (Doolan, 1996; D. L. Doolan, unpublished; C. Dobario, unpublished). It is now generally accepted that although DNA immunization is effective at inducing antigen-specific cellular responses, DNA immunization induces only moderate levels of immune activation (Zavala, 2001; Pardoll, 2002). Considerable efforts have been directed at evaluating potential immune enhancement strategies for DNA vaccination. Studies in a number of model systems have now established that the immunogenicity and protective efficacy of DNA vaccines may be significantly enhanced by heterologous prime/boost regimens, using vector systems such as recombinant poxviruses or adenoviruses. However, the complexity of such recombinant technologies and immunization strategies detracts from some of the major advantages of DNA vaccine technology as compared with more conventional vaccine delivery systems, namely ease of construction, stability and lack of requirement for a cold-chain. In addition, there are safety concerns with using live, attenuated viral vectors. Finally, pre-existing immunity to recombinant viral vectors may limit the boosting potential of recombinant virus immunization and may preclude repeated use of these immunization strategies for different vaccines, and pre-existing immunity to the vector may decrease the effectiveness of recombinant viruses immunizations.

Alphaviruses have successfully been used as viral-based gene delivery vectors. These systems induce transient, high-level antigen expression, have a broad tissue host range and the ability to infect both dividing and non-dividing cells, including antigen-presenting cells, and can induce host immuno-stimulatory responses.

Alphaviruses belong to the Togaviridae family (arbovirus) and are arthropod borne. The virion is spherical and enveloped (60-70 nm diameter), and contains two envelope glycoproteins (E1, E2) and an icosahedral capsid protein (28-35 nm). The genome consists of linear single-stranded, positive-sense RNA (12 Kb, 49 s RNA, Mol. Wt. 4 million). The infectious genes for nonstructural proteins are located at the 5′ end, which is capped; the 3′ end is polyadenylated. There are two functional segments within the genome: the 5′⅔ encodes self-assembling replicase (enzymatic non-structural proteins) that synthesizes (−) RNA genome, (+) RNA genome, and sub-genomic mRNA; the 3′⅓ (subgenomic mRNA) encodes structural proteins. Each segment contains an independent promoter. Alphavirus replicons are nucleic acids derived from the full-length virus in which the genes encoding the structural proteins (capsid and envelope proteins) have been removed, rendering the replicon capable of replicating within a cell but propagation-incompetent. In alphavirus replicon expression systems, one or more genes encoding the antigen(s) of interest can be inserted after the subgenomic promoter (26S mRNA). When such a replicon is delivered as a DNA molecule to a host cell, the alphaviral replicase machinery encoded on the replicon produces a large quantity of mRNA encoding the desired antigen, and the transfected cell undergoes apoptosis and is taken up by dendritic cells, leading to enhanced antigen presentation (Ying 1999). Alternatively, the replicon can be delivered as a naked RNA molecule, or most preferably as an alphavirus replicon particle, in which the replicon is packaged in a membrane or lipid vesicle containing the alphavirus structural proteins. Alphavirus replicons are considered propagation-defective “suicide” vectors since they infect antigen-presenting cells and induce apoptosis, but are unable to revert to an infectious state. Their predilection for infecting antigen-presenting cells, including dendritic cells, and their inability to revert to an infectious state makes them very attractive and safe vaccines. Because transfected cells are destroyed and not allowed to produce antigen chronically, theoretical concerns about tolerance, autoimmunity, and integration of plasmid sequences into the host's genome are reduced and alphavirus replicons are considered to have a better safety profile than plasmid DNA vaccines.

Three main types of alphaviruses are being developed as particle-based delivery systems (stable alphavirus replicon packaging lines): Sindbis virus (SIN), Semliki Forest virus (SFV), and Venezuelan equine encephalitis virus (VEE). We have evaluated an attenuated non-propagating Venezuelan equine encephalitis (VEE) replicon vector system that has been developed to express heterologous antigens at high levels while remaining propagation defective. The vector component of the system consists of VEE replicon particles (VRP). VRP contain a VEE self-amplifying RNA (replicon) in which the structural genes of VEE are replaced by a gene of interest, and the replicon RNA is packaged into VRP in cells by supplying the structural proteins in trans. In a preferred method, replicon RNA is packaged into VRP when cells are co-transfected with both replicon RNA and two separate helper RNAs, which together encode the full complement of VEE structural proteins. In this approach, only the replicon RNA is packaged into VRP, as the helper RNAs lack the cis-acting packaging sequence required for encapsidation. Thus, VRP can infect target cells in culture or in vivo, and can express the gene of interest to high level from the subgenomic RNA. However, they are propagation defective in that they lack the critical portion of the VEE genome (i.e. the VEE structural protein genes) necessary to produce virus particles which could spread to other cells.

### SUMMARY OF THE INVENTION

The inventive subject matter relates to an immunogenic composition and method of enhancing and broadening the immunogenicity and protective immunity in humans and animals induced by any subunit or whole organism vaccine or combination of vaccines comprising immunizing with a priming immunization mixture that contains recombinant poxviruses; recombinant adenoviruses; recombinant proteins; synthetic peptides; plasmid DNA; or live, attenuated or killed organisms, or extracts thereof, and subsequently immunizing with a boosting immunization preparation of an alphavirus replicon vaccine or combination of alphavirus replicon vaccines.

### BRIEF DESCRIPTION OF THE DRAWINGS

FIG. 1 demonstrates antigen-specific antibody responses against PyCSP induced by heterologous prime/VRP boost immunization strategies, as assessed by ELISA.

FIG. 2 demonstrates parasite-specific antibody responses directed against PyCSP induced by heterologous prime/VRP boost immunization strategies, as assessed by indirect fluorescent antibody test (IFAT) against P. yoelii sporozoites.

FIG. 3 demonstrates the expression of cell phenotypic and activation markers induced by heterologous prime/PyCSP VRP boost immunization strategies, as assessed by multiparameter flow cytometry.

FIG. 4 demonstrates antigen-specific cell mediated immune responses to PyCSP induced by heterologous prime/VRP boost immunization strategies, as assessed by intracellular cytokine staining.

FIG. 5 demonstrates antigen-specific cell mediated immune responses to PyCSP induced by heterologous prime/VRP boost immunization strategies, as assessed by ELIspot.

FIG. 6 demonstrates protection against P. yoelii parasite challenge conferred by heterologous prime/VRP boost vaccination strategies with PyCSP VRPs.

### DESCRIPTION OF THE PREFERRED EMBODIMENTS

Definitions

In the context of the present application, nm means nanometer, ml means milliliter, VEE means Venezuelan Equine Encephalitis virus, HA means hemagglutinin gene, GFP means green fluorescent protein gene, IFN means gamma-interferon, FACS means fluorescence activated cell sorter, and FBS means Fetal Bovine Serum. The expression “E2 amino acid (e.g., lys, thr, etc.) number” indicates designated amino acid at the designated residue of the E2 gene, and is also used to refer to amino acids at specific residues in the E1 gene.

As used herein, the term “alphavirus” has its conventional meaning in the art, and includes the various species such as VEE, Semliki Forest Virus (SFV), Sindbis, Ross River Virus, Western Equine Encephalitis Virus, Eastern Equine Encephalitis Virus, Chikungunya, S.A. AR86 (now referred to as “AR86”, to avoid confusion with the SARS coronavirus), Everglades virus, Mucambo, Barmah Forest Virus, Middelburg Virus, Pixuna Virus, O'nyong-nyong Virus, Getah Virus, Sagiyama Virus, Bebaru Virus, Mayaro Virus, Una Virus, Aura Virus, Whataroa Virus, Banbanki Virus, Kyzylagach Virus, Highlands J Virus, Fort Morgan Virus, Ndumu Virus, and Buggy Creek Virus. The preferred alphaviruses used in the constructs and methods of the claimed invention are VEE, AR86, Sindbis (e.g. TR339, see U.S. Pat. No. 6,008,035), and SFV. The terms “5′ alphavirus replication recognition sequence” and “3′ alphavirus replication recognition sequence” refer to the sequences found in alphaviruses, or sequences derived therefrom, that are recognized by the nonstructural alphavirus replicase proteins and lead to replication of viral RNA. These are sometimes referred to as the 5′ and 3′ ends, or alphavirus 5′ and 3′ sequences. In the replicon constructs of this invention, the use of these 5′ and 3′ ends will result in replication of the RNA sequence encoded between the two ends. The 3′ alphavirus replication recognition sequence as found in the alphavirus is typically approximately 300 nucleotides in length, which contains a more well defined, minimal 3′ replication recognition sequence. The minimal 3′ replication recognition sequence, conserved among alphaviruses, is a 19 nucleotide sequence (Hill et al., Journal of Virology, 2693-2704, 1997). These sequences can be modified by standard molecular biological techniques to further minimize the potential for recombination or to introduce cloning sites, with the proviso that they must still be recognized by the alphavirus replication machinery.

The terms “alphavirus RNA replicon”, “alphavirus DNA replicon”, or collectively “alphavirus replicon” or “alphavirus vector replicon”, refer to a nucleic acid molecule expressing alphavirus nonstructural protein genes such that it can direct its own replication amplification) and comprising, at a minimum, 5′ and 3′ alphavirus replication recognition sequences, coding sequences for alphavirus nonstructural proteins, a heterologous gene encoding an antigen and the means for expressing the antigen, and a polyadenylation tract. In the case of the alphavirus DNA replicon, the nucleic acid molecule also contains a 5′ promoter which can initiate transcription from the DNA in vivo (that is, within the subject to which the DNA replicon is administered). Johnston et al. and Polo et al. (cited previously herein) describe numerous constructs for such alphavirus RNA and DNA replicons, and such constructs are incorporated herein by reference. Specific embodiments of the alphavirus replicons utilized in the claimed invention may contain one or more attenuating mutations, an attenuating mutation being a nucleotide deletion, addition, or substitution of one or more nucleotide(s), or a mutation that comprises rearrangement or chimeric construction which results in a loss of virulence in a live virus containing the mutation as compared to the appropriate wild-type alphavirus. Examples of locations for suitable attenuating mutations in the alphavirus VEE include the following: nucleotide 3, E2-76, preferably lysine, arginine, or histidine; E2-120, preferably lysine; E2-209, preferably lysine, arginine or histidine; E1-81, preferably isoleucine, E1-253 preferably serine; E1-272, preferably threonine or serine; and E3-56 to 59, preferably a deletion of all four amino acids. Mutations may also be introduced into the alphavirus genome to improve its functionality as a vaccine vector, e.g. a mutation in the region of E2 158-162, particularly E2-160 in the Sindbis strain to enhance its targeting to dendritic cells (see for example, International PCT Publication No. WO 01/81609 Polo et al., published Nov. 1, 2001).

The terms “alphavirus structural protein/protein(s)” refers to one or a combination of the structural proteins encoded by alphaviruses. These are produced by the virus as a polyprotein and are represented generally in the literature as C-E3-E2-6k-E1. E3 and 6k serve as membrane translocation/transport signals for the two glycoproteins, E2 and E1.

Thus, use of the term E1 herein can refer to E1, E3-E1, 6k-E1, or E3-6k-E1, and use of the term E2 herein can refer to E2, E3-E2, 6k-E2, or E3-6k-E2.

“Alphavirus replicon particle”, “recombinant alphavirus particles” or “alphavirus vector particle”, used interchangeably herein, mean a virion-like structural complex incorporating an alphavirus RNA replicon that expresses one or more heterologous RNA sequences. Typically, the virion-like structural complex includes one or more alphavirus structural proteins embedded in a lipid envelope enclosing a nucleocapsid that in turn encloses the RNA. The lipid envelope is typically derived from the plasma membrane of the cell in which the particles are produced. Preferably, the alphavirus replicon RNA is surrounded by a nucleocapsid structure comprised of the alphavirus capsid protein, and the alphavirus glycoproteins are embedded in the cell-derived lipid envelope. The alphavirus replicon particles are infectious but propagation-defective, i.e. the replicon RNA contained within the particle can replicate within the host cell that the particle infects, but it cannot direct the synthesis of additional replicon particles that could infect new host cells.

As used herein, the term “antigen” means an immunogenic peptide or protein which induces an immune response (see below) to a malarial pathogen capable of infecting a mammal. Antigens suitable for use in the present invention include, but are not limited to, the following Plasmodium genes: PfCSP, PFEXP1, PfSSP2, PfLSA-1, PfLSA-3, PfMSP-1, PfAMA-1, PfEBA-175, PfMSP-3, PfMSP-4, PfMSP-5, PfRAP-1, PfRAP-2, or other novel antigens defined by the Plasmodium falciparum genomic DNA sequence [Gardner, M. J. et al. (2002) Nature 419, 498-511], or their P. vivax, P. ovale or P. malariae othologues. The entire sequence of the Plasmodium falciparum parasite is known, [dner, M. J. et al. (2002) Nature 419, 498-511]] and any of the proteins encoding by this genome could theoretically function as antigens in this invention. The term “antigen” is further intended to encompass peptide or protein analogs of known or wild-type antigens such as those described above, which analogs may be more soluble or more stable than wild type antigen, and which may also contain mutations or modifications rendering the antigen more immunologically active. Further peptides or proteins which have sequences homologous with a desired antigen's amino acid sequence, where the homologous antigen induces an immune response to the respective pathogen, are also useful. “Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g. five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGCG5′ share 50% homology. By the term “substantially homologous” as used herein, is meant DNA or RNA which is about 50% homologous, more preferably about 70% homologous, even more preferably about 80% homologous and most preferably about 90% homologous to the desired nucleic acid. Genes which are homologous to the desired antigen-encoding sequence should be construed to be included in the invention provided they encode a protein or polypeptide having a biological activity substantially similar to that of the desired antigen.

Analogs of the antigens described herein can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Modifications (which do not normally alter primary sequence) include in vivo, or in vitro chemical derivatization of polypeptides, e.g., acetylation, or carboxylation. Also included as antigens are proteins modified by glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also included as antigens according to this invention are sequences which have phosphorylated amino acid residues, e.g., phosphotyrosine, phosphoserine, or phosphothreonine. Also included as antigens are polypeptides which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The antigens of the invention are not limited to products of any of the specific exemplary processes listed herein.

The term “immune response” or “immunization” refer to the development in a subject of a humoral and/or cellular immunological response to an antigen that has been administered to the subject by the methods of this invention. “Humoral” immune responses refer to the production of antibodies, and a “cellular” immune response refers to the activation of T-lymphocytes, particularly cytolytic T-cells (“CTLs”) and helper T-cells. Specific T-cells involved in the cellular immune response include CD4⁺ and CD8⁺ T-cells.

To stimulate the humoral arm of the immune system, i.e. the production of antigen-specific antibodies, an “immunogenic fragment” will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains immunogenic activity, as measured by an assay, such as the ones described herein.

Regions of a given polypeptide that include an “epitope” can be identified using any number of epitope mapping techniques, well known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al., Proc. Natl. Acad. Sci USA (1981) 78:3824-3828) for determining antigenicity profiles and the Kyte-Doolittle technique (Kyte et al., J. Mol. Biol. (1982) 157:105-132) for hydropathy plots.

Generally, T-cell epitopes which are involved in stimulating the cellular arm of the subject's immune system, are short peptides of 8-25 amino acids, and these are not typically predicted by the above-described methods for identifying humoral epitopes. A common way to identify T-cell epitopes is to use overlapping synthetic peptides and analyze pools of these peptides, or the individual ones, that are recognized by T cells from animals that are immune to the antigen of interest, using an enzyme-linked immunospot assay (ELISPOT). These overlapping peptides can also be used in other assays such as the stimulation of cytokine release or secretion, or by the ability to interact with major histocompatibility (MHC) tetramers. Such immunogenic fragments can also be identified based on their ability to stimulate lymphocyte proliferation in response to stimulation by various fragments from the antigen of interest. The term “epitope” as used herein refers to a sequence of at least about 3 to 5, preferably about 5 to 10 or 15, and not more than about 1,000 amino acids (or any integer therebetween), which define a sequence that by itself or as part of a larger sequence, binds to an antibody generated in response to such sequence or stimulates a cellular immune response. There is no critical upper limit to the length of the immunogenic fragment, which may comprise nearly the full-length of the protein sequence, or even a fusion protein comprising two or more epitopes from a single or multiple malarial parasite proteins. An epitope for use in the subject invention is not limited to a polypeptide having the exact sequence of the portion of the parent protein from which it is derived. Indeed, there are many known species of Plasmodium and the parasite retains the ability to continue to adapt, and there are several variable domains in the parasite that exhibit relatively high degrees of variability between species. Thus the term “epitope” encompasses sequences identical to the native sequence, as well as modifications to the native sequence, such as deletions, additions and substitutions (generally conservative in nature).

Immunization Methods

One component of the methods and compositions of the present invention is the use of a “priming” immunization, comprising the initial administration of one or more antigens to an animal, especially a human patient, in preparation for subsequent administration(s) of the same antigen. Specifically, the term “priming”, or alternatively “initiating” or “activating” an immune response or “enhancing” and “potentiating”, as used herein, defines a first immunization using an antigen which induces an immune response to the desired antigen and recalls a higher level of immune response to the desired antigen upon subsequent re-immunization with the same antigen when administered in the context of the same or a different vaccine delivery system. Specifically as used in this application, a “priming immunization” refers to the administration of a composition comprising a preparation containing a malarial antigen. As used herein, a “priming immunogenic composition or preparation” refers to a preparation containing a malarial antigen or fragment thereof with the preparation being selected from the group consisting of: a recombinant virus expression system; a recombinant protein antigen or a recombinant polypeptide; a synthetic peptide; a polynucleotide vector; a whole organism or extract and combinations thereof.

Another component of the methods and compositions of the present invention is the use of a “boosting immunization”, or a “boost”, which means the administration of a composition delivering the same malarial antigen as encoded in the priming immunization, but utilizing a composition that contains an alphavirus replicon. A boost is sometimes referred to as an anamnestic response, i.e. an immune response in a previously sensitized animal. A boosting immunization or a boosting immunogenic composition can comprise multiple doses, which may be the same or different amounts.

As used in this application, a boosting immunization or a boosting immunogenic composition can comprise one, two, three or multiple doses.

The widespread deployment of subunit vaccines may depend upon the use of immunization regimes in which the vaccine is administered together with recombinant alphaviruses (replicons of Venezulan Encephalitis Virus, Semliki Forest virus or Sindbis virus, for instance, and others like them) recombinant poxviruses (canarypox, cowpox, fowlpox, monkeypox, for instance, and others like them); recombinant adenoviruses or adeno-associated virus; recombinant proteins; synthetic peptides; plasmid DNA; or live, attenuated or killed organisms, or extracts thereof. We have determined that boosting with an alphavirus replicon vaccine containing malaria immunogens following priming with a heterologous vaccine such as recombinant poxviruses; recombinant adenoviruses; recombinant proteins; synthetic peptides; plasmid DNA, or live, attenuated or killed organisms, or extracts thereof, provides a method of immunization that induces a broad immunogenic response to the encoded antigen and protective immunity against parasite challenge. As a result of this method, the amount (dose) of vaccine needed to achieve protective immunity may be reduced, the duration of vaccine-induced protective immunity may be increased, and the vaccine coverage in a vaccinated population may be broadened.

The Alphavirus genus includes a variety of viruses, all of which are members of the Togaviridae family. The alphaviruses include Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, Semliki Forest virus, Middleburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus, and Buggy Creek virus. The viral genome is a single-stranded, messenger-sense RNA, modified at the 5′-end with a methylated cap and at the 3′-end with a variable-length poly (A) tract. Structural subunits containing a single viral protein, capsid, associate with the RNA genome in an icosahedral nucleocapsid. In the virion, the capsid is surrounded by a lipid envelope covered with a regular array of transmembrane protein spikes, each of which consists of a heterodimeric complex of two glycoproteins, E I and E2. See Pedersen et al., J. Virol 14:40 (1974). The Sindbis and Semliki Forest viruses are considered the prototypical alphaviruses and have been studied extensively. See Schelsinger, The Togaviridae and Flaviviridae, Plenum Publishing Corp., New York (1986). The VEE virus has been studied extensively, see, e.g., U.S. Pat. No. 5,185,440.

The complete genomic sequences, as well as the sequences of the various structural and non-structural proteins are known in the art for numerous alphaviruses and include: Sindbis virus genomic sequence (GenBank Accession Nos. J02363, NCBI Accession No. C_{—}001547), S.A.AR86 genomic sequence (GenBank Accession No. U38305), VEE genomic sequence (GenBank Accession No. L04653, NCBI Accession No. NC_{—}001449), Girdwood S. A. genomic sequence (GenBank Accession No. U38304), Semliki Forest virus genomic sequence (GenBank Accession No. X04129, NCBI Accession No. NC_{—}003215), and the TR339 genomic sequence (Klimstra et al., (1988) J. Virol. 72:7357; McKnight et al., (1996) J. Virol. 70:1981); the disclosures of which are incorporated herein by reference in their entireties.

The studies of these viruses have led to the development of techniques for vaccinating against diseases through the use of alphavirus vectors for the introduction of genes expressing antigens derived from the organism(s) causing disease. The alphavirus replicon system, as described in U.S. Pat. No. 6,190,666 to Garoff et al., U.S. Pat. Nos. 5,792,462 and 6,156,558 to Johnston et al., U.S. Pat. Nos. 5,814,482, 5,843,723, 5,789,245, 6,015,694, 6,105,686 and 6,376,236 to Dubensky et al; U.S. Published Application No. 2002/0015945 A1 (Polo et al.), U.S. Published Application No. 2001/0016199 (Johnston et al.), Frolov et al. (1996) Proc. Natl. Acad. Sci. USA 93:11371-11377 and Pushko et al. (1997) Virology 239:389-401, is particularly well-suited as a vector for delivering disease organism antigens. The alphavirus replicon vector system can be delivered in the form of DNA replicons, which are “launched” within the host cell to express the replicon RNA which then expresses the antigen. Similarly, naked RNA replicons can be introduced directly into the host cell, e.g. by transfection of patient's cells in an ex vivo manner. Alternatively, the replicon can be delivered to the subject as an alphavirus replicon particle. In all of these embodiments, the replicon RNA contains sequences required for replication and packaging of the RNA into a virus-like particle. It contains a nonstructural proteins open reading frame (ORF), which provides viral protein required for genome replication and transcription of subgenomic RNA, but lacks the structural protein genes necessary for formation of viral particle. The replicon is engineered so that the subgenomic RNA contains ORF(s) coding for a gene of interest, in this invention one or more genes encoding malarial antigens. To produce the alphavirus replicon particles, one or more helper nucleic acids provide the alphavirus capsid and glycoprotein genes. The replicon RNA vector and the one or more helper nucleic acids are introduced into an alphavirus-permissive cell, the replicon RNA is packaged into virus-like particles, which are harvested and purified from these cells to produce an immunogenic preparation, i.e. a vaccine composition.

Immunogenic Composition Dosage and Routes of Administration

Pharmaceutical formulations of the boosting immunization is an amount sufficient to evoke an immune response in the subject to which the pharmaceutical formulation is administered. The boosting immunization preparation comprises at least one alphavirus replicon containing an antigen or fragment thereof and a priming immunization preparation comprising a preparation containing antigen or fragment thereof, the preparation being selected from the group consisting of: a recombinant virus expression system; a recombinant protein antigen or a recombinant polypeptide; a synthetic polypeptide; a polynucleotide vector; a whole organism or extract or combinations thereof. The immunogenic preparations are administered in a manner compatible with the dosage formulation, and in such amount as will be prophylactically and/or therapeutically effective. An amount of from about 10⁴ to about 10⁹ infectious units of alphavirus replicon particles (ARPs) or VRPs per dose is believed suitable, depending upon the subject to be treated, the route by which the VRPs are administered, the immunogenicity of the expression product, the types of effector immune responses desired, and the degree of protection desired. Precise amounts of the active ingredient required to be administered may depend on the judgment of the physician, veterinarian or other health practitioner and may be peculiar to each individual, but such a determination is within the skill of such a practitioner.

Exemplary pharmaceutically acceptable carriers include, but are not limited to, sterile pyrogen-free water and sterile pyrogen-free physiological saline solution. Immunogenic compositions comprising the VRPs may be formulated by any of the means known in the art. Such compositions, especially vaccines, are typically prepared as injectables, either as liquid solutions or suspensions. Solid forms suitable for solution in, or suspension in, liquid prior to injection may also be prepared.

The active immunogenic ingredients (e.g. VRPs) are often mixed with excipients or carriers which are pharmaceutically acceptable and compatible with the active ingredient. Suitable excipients include but are not limited to sterile water, saline, dextrose, glycerol, ethanol, or the like and combinations thereof, as well as stabilizers, e.g. Human Serum Albumin (HSA) or other suitable proteins and reducing sugars.

In addition, if desired, the vaccines may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3hydroxyphosphoryloxy)-ethylamine (CGP 19835A, referred to as MTP-PE); MF59 (see International Publication No. WO90/14837); RIBI (Corixa, Seattle Wash.), which contains three components extracted from bacteria, monophosphoryl lipid A, trehalose dimycolate and cell wall skeleton (MPL+TDM+CWS) in a 2% squalene/Tween 80 emulsion; and saponins, e.g. Stimulon® (Cambridge Bioscience, Worcester, Mass.). The effectiveness of an adjuvant may be determined by measuring the amount of antibodies directed against the malarial antigen resulting from administration of the immunizing preprations of this invention which are also comprised of one or more adjuvants. Such additional formulations and modes of administration as are known in the art may also be used.

Subjects which may be administered the priming and boosting pharmaceutical formulations of this invention include human and animal (e.g., dog, cat, cattle, horse, donkey, mouse, hamster, monkeys, guinea pigs, birds, eggs) subjects.

Each of the priming and boosting immunizations of this invention may be given in a single dose or multiple dose schedule. A multiple dose schedule is one in which a priming immunization may include, at least one dose, for example, 1 to 3 separate doses, followed by one or more boosting immunizations, for example, 1 to 3 separate doses, administered at subsequent time intervals as required to induce, maintain, enhance and/or reinforce the immune response, e.g., weekly or at 1 to 4 months, and if needed, a subsequent dose(s) after several weeks, months or years. Each priming or boosting preparation may express either full-length malarial antigen, an immunogenic fragment thereof, an epitope derived from the malarial antigen, or a combination thereof.

All references cited in the present application are incorporated by reference herein to the extent that there is no inconsistency with the present disclosure.

In the P. yoelii rodent malaria model, PyCSP and PyHEP17 are target antigens of protective CD8+ and CD4+ T cell responses and protective antibody responses. Therefore, recombinant PyCSP and PyHEP17 alphavirus vaccines in mice provided an ideal experimental system with which to measure the enhancing effect of boosting with one or more recombinant alphavirus vaccines subsequent to a priming immunization with a heterologous vaccine. Some examples of different immunization regimes are depicted in the specific examples.

Having described the invention, the following examples are given to illustrate specific applications of the invention including the best mode now known to perform the invention. These specific examples are not intended to limit the scope of the invention described in this application.

### WORKING EXAMPLES

### Example 1

Boosting of Parasite-Specific and Antigen-Specific Antibody Responses by VEE Viral Replicon Particles in Heterologous Prime/VRP Boost Immunization Strategies in a Mouse Model of Malaria.

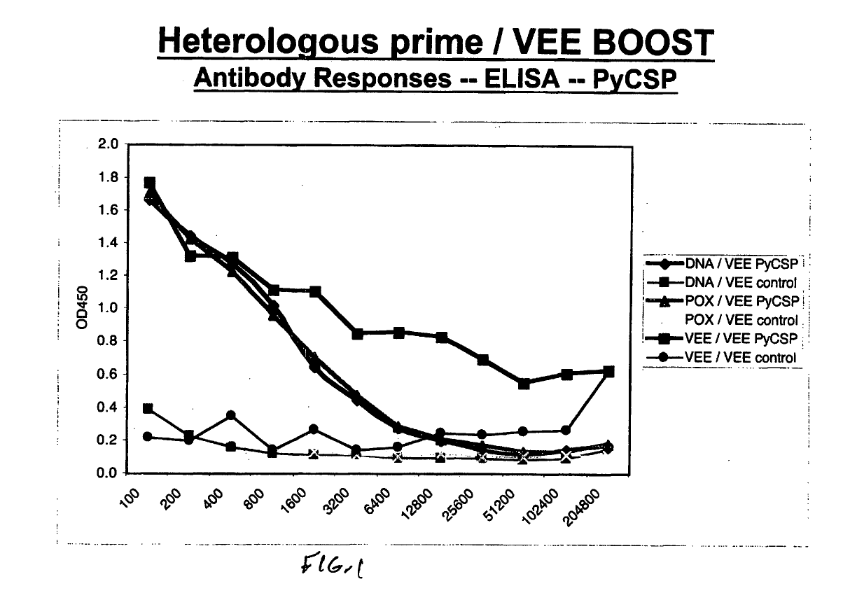

FIG. 1 demonstrates antigen-specific antibody responses against PyCSP induced by heterologous prime/VRP boost immunization strategies, as measured by ELISA.

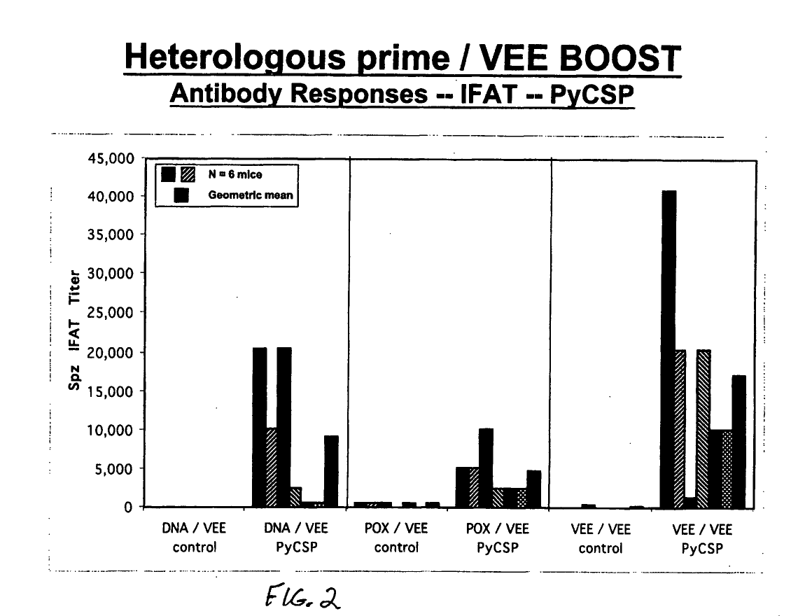

FIG. 2 demonstrates parasite-specific antibody responses directed against PyCSP induced by heterologous prime/VRP boost immunization strategies, as measured by indirect fluorescent antibody test (IFAT) against P. yoelii sporozoites.

Study:

Female 4- to 8-wk-old AnNCr (H-2d) mice were obtained from The National Cancer Institute (Charles River Laboratories, Fredrick, Md.). In homologous VRP immunization regimens, groups of 6 mice were primed by 1M immunization with 1×10⁶ VEE viral replicon particles expressing PyCSP and then boosted 6 weeks later with VEE viral replicon particles expressing PyCSP or an unrelated control gene (influenza HA). In heterologous prime/VRP boost immunization regimens, groups of 6 mice were primed by 1M immunization with 1×10⁶ VEE viral replicon particles expressing PyCSP and then boosted 6 weeks later 1M with plasmid DNA encoding PyCSP or control DNA (100 ug), recombinant POXvirus expressing PyCSP or control POXvirus (1×10⁷ pfu) or recombinant ADENOvirus expressing PyCSP or control ADENOvirus (1×10⁸ particles). For intramuscular injections, each tibialis anterior muscle was injected with half the vaccine dose in a volume of 50 μl per muscle, using a 0.3 ml insulin syringe fitted with a plastic collar cut from a micropipette tip, adjusted to limit the needle penetration to a distance of about 2 mm into the muscle. Sera were collected 2-3 weeks after each priming or boosting immunization, and individual sera were assayed for antigen-specific antibodies by ELISA against PyCSP recombinant protein or synthetic peptide (FIG. 1) or parasite-specific antibody responses by indirect fluorescent antibody test (IFAT) against P. yoelii sporozoites (FIG. 2).

Methods:

Antibody ELISA assays: Mice were bled via the tail vein approximately 2-3 wk after each priming or boosting immunization. Antibodies were measured by enzyme-linked immunosorbent assay (ELISA) against recombinant PyCSP protein or synthetic peptides representing the immunodominant B cell epitope on PyCSP or PyHEP17, as previously described (Charoenvit et al., 1987; 1999). Briefly, 50 μl of 0.1 μg/ml of recombinant protein in PBS was added into wells of Immunolon II ELISA plates (Dynatech Laboratory Inc., Chantilly, Va.) and incubated for 6 h at room temperature. Wells were washed 4 times with PBS containing 0.05% Tween 20 (washing buffer) and incubated overnight at 4° C. with 100 μl of 5% nonfat dry milk in PBS (blocking buffer). After washing 4 times with washing buffer, the wells were incubated for 2 hr with 50 μl of 2-fold serial dilutions of mouse serum or a 1:20 dilution of supernatant mAb NYS1 (for PyCSP) diluted in PBS containing 3% nonfat dry milk (diluting buffer). The wells were again be washed 4 times, incubated for 1 hr with peroxidase-labeled goat anti-mouse IgG (Kirkegaad & Perry, Gaithersburg, Md.) diluted 1:2000 in diluting buffer, then again washed 4 times. The wells were incubated for 20 min with 100 μl of a solution containing ABTS substrate [2,2′-azino-di-(3 ethylbenzthiazoline sulfonate] (Kirkegaard & Perry, Gaithersburg, Md.) and H₂O₂. Color reaction was measured in a micro-ELISA automated reader at OD 410 nm. All reaction steps except blocking were performed at room temperature. ELISA data are presented as the mean OD reading for each reciprocal serum dilution of sera collected from individual mice (n=6) after the third immunization.

Antibody IFAT assays: Immunofluorescence assays were carried out using air-dried P. yoelii sporozoites as previously described (Charoenvit et al., 1987; 1999). IFAT data are presented as the reciprocal of the last serum dilution at which fluorescence was scored as positive for individual sera collected from mice (n=6 per group) after the third immunization, and the mean of the group.

Data presented in FIG. 1 demonstrate antigen-specific antibody responses induced by homologous immunization with PyCSP viral replicon particles or by heterologous immunization with DNA prime/VRP boost or POXvirus prime/VRP boost regimens, as measured in sera collected after the boost by ELISA against recombinant PyCSP protein or PyCSP synthetic peptide as capture antigen.

Data presented in FIG. 2 demonstrate parasite-specific antibody responses induced by homologous immunization with PyCSP viral replicon particles or by heterologous immunization with DNA prime/VRP boost or POXvirus prime/VRP boost regimens, as measured in sera collected after the boost by an indirect fluorescent antibody test (IFAT) against P. yoelii sporozoites.

Results represented in FIGS. 1 and 2 show that VEE viral replicon particles expressing an antigen of interest can effectively boost antibody responses directed against malaria parasites or parasite antigen in heterologous prime/VRP boost vaccination strategies.

### Example 2

Boosting of Antigen-Specific Cell Mediated Immune Responses by VEE Viral Replicon Particles in Heterologous Prime/VRP Boost Immunization Strategies in a Mouse Model of Malaria

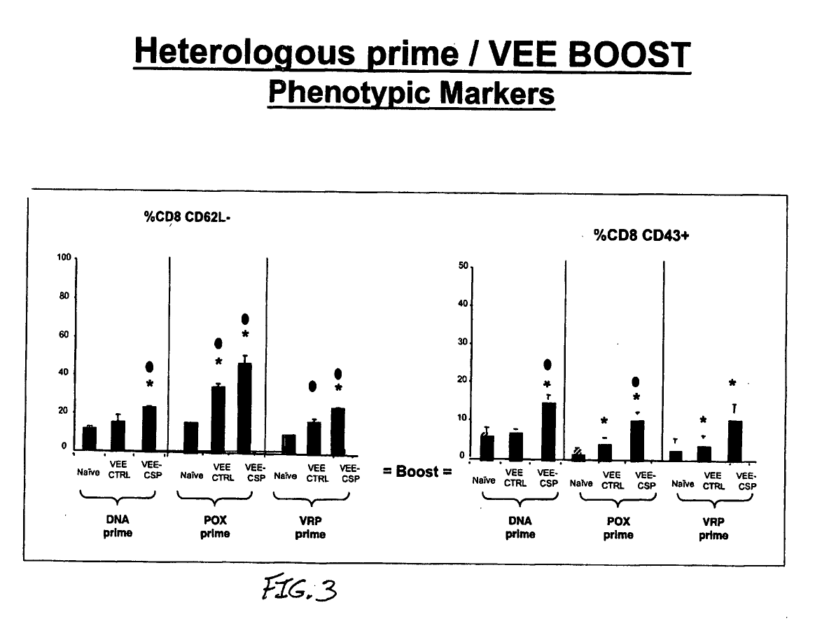

FIG. 3 demonstrates the expression of cell phenotypic and activation markers induced by heterologous prime/VRP boost immunization strategies, as assessed by multiparameter flow cytometry.

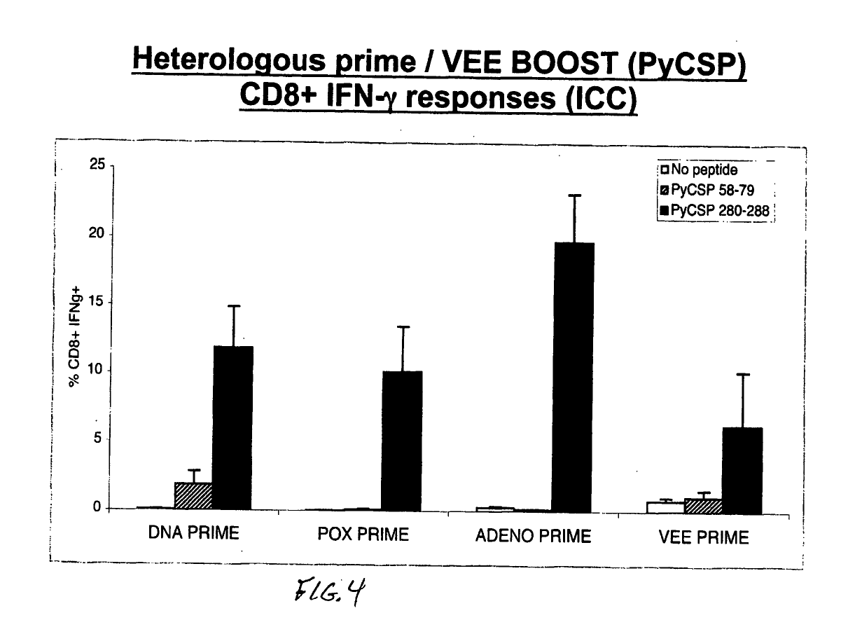

FIG. 4 demonstrates antigen-specific cell mediated immune responses to PyCSP induced by heterologous prime/VRP boost immunization strategies, as assessed by intracellular cytokine staining.

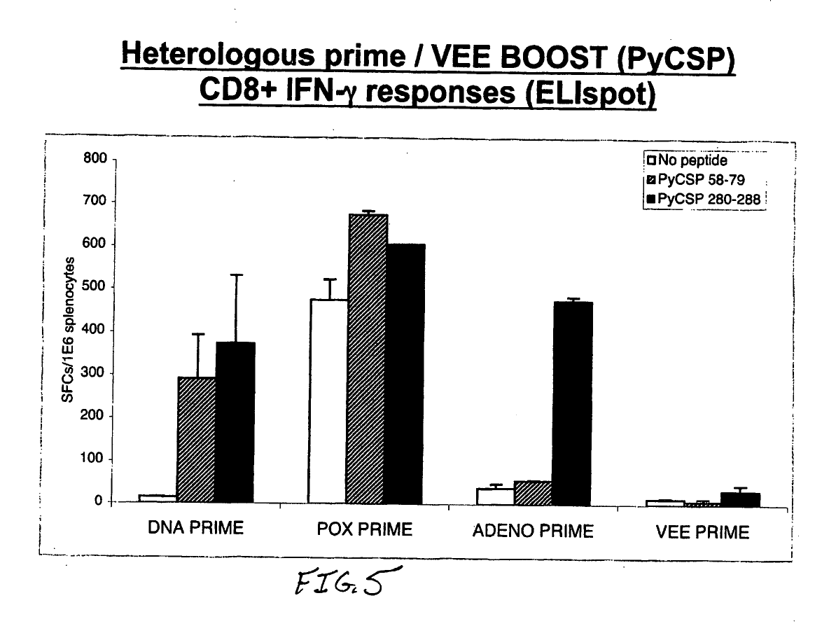

FIG. 5 demonstrates antigen-specific cell mediated immune responses to PyCSP induced by heterologous prime/VRP boost immunization strategies, as assessed by IFNg ELIspot.

Study:

Female 4- to 8-wk-old AnNCr (H-2d) mice were obtained from The National Cancer Institute (Charles River Laboratories, Fredrick, Md.). In homologous VRP immunization regimens, groups of 6 mice were primed by 1M immunization with 1×10⁶ VEE viral replicon particles expressing PyCSP and then boosted 6 weeks later with VEE viral replicon particles expressing PyCSP or an unrelated control gene (influenza HA). In heterologous prime/VRP boost immunization regimens, groups of 6 mice were primed by 1M immunization with 1×10⁶ VEE viral replicon particles expressing PyCSP and then boosted 6 weeks later 1M with plasmid DNA encoding PyCSP or control DNA (100 ug), recombinant POXvirus expressing PyCSP or control POXvirus (1×10⁷ pfu) or recombinant ADENOvirus expressing PyCSP or control ADENOvirus (1×10⁸ particles). Phenotypic changes in bulk CD8+ or CD4+ T cell populations and expression of cell surface activation markers were evaluated at 3 days or 7 days (FIG. 3) following the boost. Splenocytes were harvested at 2 weeks after the boost, and assayed for antigen specific cell mediated immune responses by IFNg ELIspot (FIG. 4 or intracellular cytokine staining flow cytometry assays (FIG. 5).

Methods:

Intracellular cytokine staining and FACS analysis: A20.2J cells were pulsed with or without synthetic peptides representing immunodominant or subdominant CD8+ or CD4+ T cell epitopes derived from PyCSP (10 μg/ml) for 1 hr at 37° C. in 5% CO₂, and irradiated in a ¹³⁷CS gamma irradiator (16,000 rads). Then, 100 μl/well of spleen cells (5×10⁶ cells/ml) and 100 μl/well of A20.2J cells (1.5×10⁶ cells/ml) pulsed with or without PyCSP peptide were incubated in duplicates, triplicates or quadruplicates in U-bottom 96-well plates (Costar) in the presence of 1 μM Brefeldin A (GolgiPlug™, Pharmingen, San Diego, Calif.) in an atmosphere of 5% CO₂ at 37° C. for 16 h. Plates were spun at 1,200 rpm for 5 min, the supernatant flicked, and the cell pellet resuspended by gentle vortexing. Cell surface markers were stained with 0.3-0.5 μl/well of anti-CD8-APC, anti-CD4-PERCP, or anti-CD62L, anti-CD69, anti-CD25 anti-CD43, anti-CD71, or anti-CD152 Abs (Pharmingen, San Diego, Calif.) in a final volume of 100 μl in FACS wash, on ice in the dark for 20 min. After the surface staining, cells were washed twice with FACS wash, gently resuspended, and incubated with 90 μl of Perm/Fix buffer (Pharmingen, San Diego, Calif.) for 20 min on ice in the dark. Next, cells were washed with 100 μl of Perm/Wash buffer and intracellular IFN-γ or TNF-α were stained with 0.5 μl/well of anti-IFN-γ-PE or anti-TNF-α-PE Abs (Pharmingen, San Diego, Calif.) in a final volume of 100 μl in Perm/Wash buffer. After 20 min incubation on ice in the dark, cells were washed twice with Perm/Wash, once with FACS wash, resuspended in 100 μl of FACS wash and stored at 4° C. prior to analysis. The frequency of cells secreting IFN-γ and TNF-α was determined by four-color fluorescent activated cell sorting using the FACSCalibur™ (Becton Dickinson Immunocytometry Systems, San Jose, Calif.).

Interferon γ ELISPOT: Multiscreen MAHAS 4510 plates (Millipore, Bedford, Mass.) were coated with 60 μl/well of sterile carbonate/bicarbonate buffer containing 10 μg/ml of anti-murine IFN-γ (R4, Pharmingen, San Diego, Calif.) and incubated overnight at room temperature. Plates were washed twice with 200 μl/well RPMI medium and twice with cRPMI medium containing Penicillin/Streptomycin, L-Glutamine and 10% FBS, and incubated with 200 μl/well of cRPMI medium in 5% CO₂ at 37° C. for at least 3 hr. After blocking, the plates were washed once more with cRPMI before the addition of target and effector cells. A20.2J (e.g., ATCC clone HB-98) or P815 (ATCC TIB 64) target cells were washed once with cRPMI, incubated at 5×10⁶ cells/ml with or without synthetic peptides representing immunodominant or subdominant CD8+ or CD4+ T cell epitopes derived from PyCSP or PyHEP17 (10 g/ml) for 1 hr at 37° C. in 5% CO₂, and irradiated in a ¹³⁷CS gamma irradiator (A20.2J at 16,000 rads and P815 at 10,000 rads). Next, target cells were washed 3 times with cRPMI, diluted to 1.0×10⁶ cells/ml (P815) or 1.5×10⁶ cells/ml (A20.2J) in cRPMI. To obtain splenocytes, immunized mice were sacrificed 2-7 wks after the final immunization (3-6 mice/group), their spleens removed to a sterile tissue screen and ground with glass pestle into a sterile petri dish using cRPMI. The spleen cell suspensions were washed 3 times, counted and diluted to 5×10⁶ cells/ml and 2.5×10⁶ cells/ml. Both spleens and target cells were plated in quadruplicates at 100 μl/well, and incubated in 5% CO₂ at 37° C. for 36 h. Plates were washed 3 times with PBS followed by 4 times with PBS-T (PBS 0.05% Tween20). Then, 100 μl/well of biotinylated anti-IFN-γ (XMG1.2, Pharmingen, San Diego, Calif.) at 2 μg/ml in PBS-T was added to each well and the plate was incubated overnight at 4° C. Plates were washed 6 times with PBS-T and then 100 μl/well peroxidase conjugated streptavidin (Kirkekaard & Perry, Gaithesburg, Md.) was added at 1:800 dilution in PBS-T. After 1 hr incubation at room temperature, plates were washed 6 times with PBS-T followed by 3 times with PBS alone, and developed with DAB reagent (Kirkekaard & Perry, Gaithesburg, Md.) according to manufacturer's instructions. After 15 min, the plates were rinsed extensively with dH₂O to stop the colorimetric substrate, dried and stored in the dark. Spots were counted using an automated KS Elispot reader (Carl Zeiss Vision, Germany).

Data presented in FIG. 3 demonstrate the expression of phenotypic and activation markers induced by induced by homologous immunization with PyCSP viral replicon particles or by heterologous immunization with DNA prime/VRP boost or POXvirus prime/VRP boost regimens, as assayed by multiparameter flow cytometry. Also depicted is the expression of phenotypic and activation markers in naïve mice. Phenotypic changes in bulk CD8+ or CD4+ T cells were evaluated at 3 days or 7 days following boost. Responses at 7 days post immunization are presented, for CD62L (expressed by naïve T cells) and CD43 (marker for activated CD8 CTL) expressed on gated CD8+ T cells. Not presented are data showing expression of CD69 (early activation marker), CD71 (transferin receptor, activation marker), CD25 (IL-2 receptor, expressed on activated & proliferating cells), and CD152 (CTLA-4, activation marker).

Data presented in FIG. 4 demonstrate antigen-specific cytokine (IFNg) responses induced by homologous immunization with PyCSP viral replicon particles or by heterologous immunization with DNA prime/VRP boost or POXvirus prime/VRP boost regimens, as assayed using splenocytes collected after the boost as effector cells in intracellular cytokine flow cytometry (ICC, CD8+ gated population) assays with MHC-matched target cells expressing class I and class II (A20.2J cells) pulsed with synthetic peptides representing defined PyCSP CD8+ and/or CD4+ T cell epitopes or without peptide. PyCSP residues 280-288=dominant CD8+ T cell epitope; PyCSP residues 280-295, overlapping dominant CD4+ and dominant CD8+ T cell epitope; PyCSP residues 57-70=dominant CD4+ T cell epitope; PyCSP residues 58-67=subdominant CD8+ T cell; and PyCSP residues 58-79=overlapping dominant CD4+ T cell epitope and subdominant CD8+ T cell epitope. Data show results with pooled splenocytes (n=6) collected after the boost. ICC data are presented as the frequency of CD8+ T cells secreting IFNg.

Data presented in FIG. 5 demonstrate antigen-specific cytokine (IFNg) responses induced by homologous immunization with PyCSP viral replicon particles or by heterologous immunization with DNA prime/VRP boost or POXvirus prime/VRP boost regimens, as assayed using splenocytes collected after the boost as effector cells in IFNg ELIspot assays with MHC-matched target cells expressing class I and class II (A20.2J cells) pulsed with synthetic peptides representing defined PyCSP CD8+ and/or CD4+ T cell epitopes or without peptide. PyCSP residues 280-288=dominant CD8+ T cell epitope; PyCSP residues 280-295, overlapping dominant CD4+ and dominant CD8+ T cell epitope; PyCSP residues 57-70 dominant CD4+ T cell epitope; PyCSP residues 58-67=subdominant CD8+ T cell; and PyCSP residues 58-79=overlapping dominant CD4+ T cell epitope and subdominant CD8+ T cell epitope. Data show results with pooled splenocytes (n=6) collected after the boost. ELIspot data are plotted as spot forming cells (SFC) per million splenocytes.

Results represented in FIGS. 3, 4 and 5 show that VEE viral replicon particles expressing an antigen of interest can effectively boost cell mediated immune responses directed against malaria antigens in heterologous prime/VRP boost immunization strategies.

### Example 3

Protection Against Parasite Challenge by Heterologous Prime/VRP Boost Immunization Strategies in a Mouse Model of Malaria.

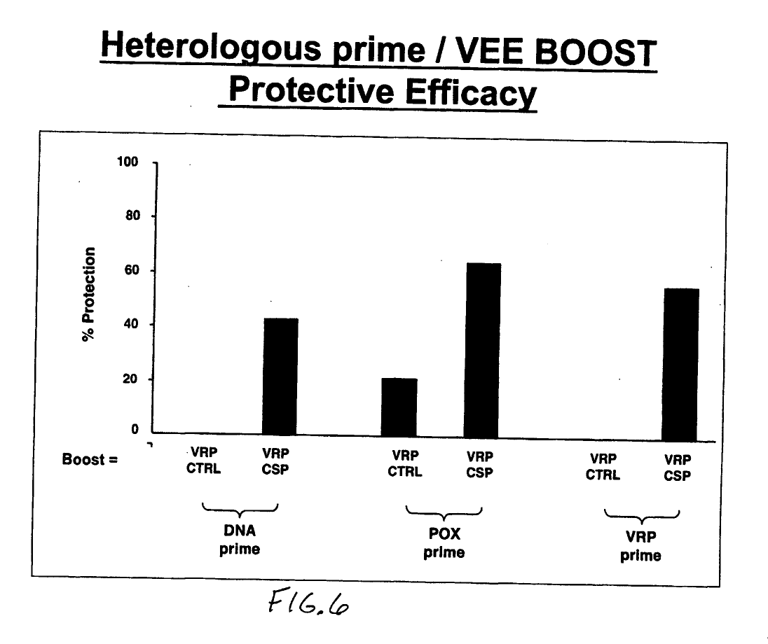

FIG. 6 demonstrates protection against P. yoelii parasite challenge conferred by heterologous prime/VRP boost vaccination strategies with PyCSP VRPs.

Study:

Female 4- to 8-wk-old AnNCr (H-2d) mice were obtained from The National Cancer Institute (Charles River Laboratories, Fredrick, Md.). In homologous VRP immunization regimens, groups of 12-14 mice were primed by 1M immunization with 1×10⁶ VEE viral replicon particles expressing PyCSP and then boosted 6 weeks later with VEE viral replicon particles expressing PyCSP or an unrelated control gene (influenza HA). In heterologous prime/VRP boost immunization regimens, groups of 6 mice were primed by 1M immunization with 1×10⁶ VEE viral replicon particles expressing PyCSP and then boosted 6 weeks later 1M with plasmid DNA encoding PyCSP or control DNA (100 ug), recombinant POXvirus expressing PyCSP or control POXvirus (1×10⁷ pfu) or recombinant ADENOvirus expressing PyCSP or control ADENOvirus (1×10⁸ particles). Mice (n=12-14 per group) were challenged with infectious P. yoelli sporozoites (100 sporozoites) at 2 weeks after the boost, and followed for 14 days for the presence or absence of blood-stage parasitemia. Protection was defined as the complete absence of blood-stage parasitemia (FIG. 6).

Methods:

Blood stage protection against challenge with Plasmodium yoelii parasites: P. yoelii (17XNL nonlethal strain, clone 1.1) parasite was maintained by alternating passage of the arasites in Anopheles stephensi mosquitoes and CD-1 mice. Sporozoites were harvested from nonirradiated P. yoelii 17XNL infected mosquitoes 14 days after an infectious blood meal by hand-dissection. Mice (n=12 per group) were challenged by tail-vein injection of 100 infectious sporozoites in a 0.2 ml volume of M199 containing 5% normal mouse serum. Since it has been established previously that infection with as few as one or two sporozoites of P. yoelii 17XNL will result in patent infection of 50% of BALB/c mice (ID50), the challenge dose used here represents a virulent parasite challenge. Giemsa-stained thin blood films were examined on days 5-14 post-challenge, up to 50 oil-immersion fields being examined for parasites. Protection was defined as the complete absence of blood-stage parasitemia.

Data presented in FIG. 6 demonstrate the percentage of mice that were completely protected against development of blood-stage parasitemia (sterile protection) following challenge with infectious P. yoelii sporozoites.

Results represented in FIG. 6 show that heterologous prime/VRP boost immunization strategies comprising priming boosting with a non-alphavirus expression system and then boosting with VEE viral replicon particles expressing malaria antigens can effectively induce sterile protective immunity against malaria parasite challenge.

Summary:

Overall, data demonstrate that heterologous prime/boost immunization strategies incorporating VEE viral replicon particles expressing malaria antigens as a boost can effectively activate both humoral and cellular arms of the immune system, and induce a profile of broad epitope recognition, and can protect against parasite challenge.

Synthetic Peptides

Synthetic peptides based on PyCSP or PyHEP17 sequences used for in vitro stimulation for T cell assays were synthesized commercially at >90% purity (AnaSpec Inc., San Jose, Calif.; Research Genetics, Huntsville, Ala.)

TABLE IA
PyCSP peptides

PyCSP residues 280-295, sequence SYVPSAEQILEFVKQI: overlapping
dominant CD4+ and dominant CD8+ T cell epitope.
PyCSP residues 280-288, sequence SYVPSAEQI:
dominant CD8+ T cell epitope.
PyCSP residues 58-79, sequence YNRNIVNRLLGDALNGKPEEK:
overlapping dominant
CD4+ T cell epitope and subdominant CD8+ T cell epitope.
PyCSP residues 58-67, sequence IYNRNIVNRL: subdominant
CD8+ T cell epitope.
PyCSP residues 57-70, sequence KIYNRNIVNRLLGD: dominant
CD4+ T cell epitope.
PyCSP repeat, sequence QGPGAPQGPGAPQGPGAP: dominant B
cell epitope.

TABLE IB
PyHEP17 peptides

PyHEP residues 61-75 (#4612), sequence EEIVKLTKNKKSLRK:
dominant CD4+ T cell epitope/nested CD8+ T cell epitope.
PyHEP residues 66-80 (#4613), sequence LTKNKKSLRKINVAL:
subdominant CD4+ T cell epitope/nested CD8+ T cell epitope.
PyHEP residues 71-85 (#4614), sequence KSLRKINVALATAL:
dominant CD4+ T cell epitope/nested CD8+ T cell epitope.
PyHEP residues 73-81, sequence LRKINVALA: subdominant
CD8+ T cell epitope.
PyHEP residues 74-82, sequence RKINVALAT: subdominant
CD8+ T cell epitope.
PyHEP residues 96-110 (#4619), sequence GLVMYNTEKGRRPFQ:
subdominant CD4+ T cell epitope.
PyHEP residues 126-140 (#4625), sequence SFPMNEESPLGFSPE:
subdominant CD4+ T cell epitope.
PyHEP residues 136-150 (#4627), sequence GFSPEEMEAVASKFR:
subdominant CD4+ T cell epitope.
PyHEP residues 126-140 (MR68), sequence SFPMNEESPLGFSPE:
dominant B cell epitope.

### REFERENCES

1. Berglund et al. 1998. Enhancing immune responses using suicidal DNA vaccines. Nat. Biotechnol. 16:562.
2. Berglund et al. 1999. Immunization with recombinant Semliki Forest virus induces protection against influenza challenge in mice. Vaccine 17:497.
3. Dubensky, T. W., M. Liu, and J. B. Ulmer. 2000. Delivery systems for gene-based vaccines. Mol. Med. 6:723.
4. Hevey, M., D. Negley, P. Pushko, J. Smith, and A. Schmaljohn. 1998. Marburg virus vaccines based upon alphavirus replicons protect guinea pigs and nonhuman primates. Virology 251:28.
5. Leitner et al. 2000. Enhancement of tumor-specific immune response with plasmid DNA replicon vectors. Cancer Res. 60:51.
6. Liu, M. A., and J. B. Ulmer. 2000. Gene-based vaccines. Mol. Ther. 1:497.
7. Perkus, M. E., Limbach, K. & Paoletti, E. (1989) J. Virol. 63, 3829-3836
8. Polo et al. 1999. Stable alphavirus packaging cell lines for Sindbis virus and Semliki Forest virus-derived vectors. Proc. Natl. Acad. Sci. USA 96:4598.
9. Pushko et al. 2000. Recombinant RNA replicons derived from attenuated Venezuelan equine encephalitis virus protect guinea pigs and mice from Ebola hemorrhagic fever virus. Vaccine 19:142.
10. Ying et al. 1999. Cancer therapy using a self-replicating RNA vaccine. Nat. Med. 5:823.
11. Bojang K A, Milligan P J, Pinder M, Vigneron L, Alloueche A, Kester K E, Ballou W R, Conway D J, Reece W H, Gothard P, Yamuah L, Delchambre M, Voss G, Greenwood B M, Hill A, McAdam K P, Tornieporth N, Cohen J D, Doherty T; RTS, S Malaria Vaccine Trial Team. Efficacy of RTS,S/AS02 malaria vaccine against Plasmodium falciparum infection in semi-immune adult men in The Gambia: a randomised trial. Lancet. 2001 Dec. 8; 358 (9297):1927-34.
12. Carvalho L J, Daniel-Ribeiro C T, Goto H. Malaria vaccine: candidate antigens, mechanisms, constraints and prospects. Scand J. Immunol. 2002 October; 56 (4):327-43
13. Doolan D L, Sedegah M, Hedstrom R C, Hobart P, Charoenvit Y, Hoffman S L. Circumventing genetic restriction of protection against malaria with multigene DNA immunization: CD8+ cell-, interferon gamma-, and nitric oxide-dependent immunity. J Exp Med. 1996 Apr. 1; 183 (4):1739-46.
14. Engers, H. D. & Godal, T. Malaria vaccine development: current status. Parasitology Today 21998. 14, 56-64.
15. Epstein J E, Gorak E J, Charoenvit Y, Wang R, Freydberg N, Osinowo O, Richie T L, Stoltz E L, Trespalacios F, Nerges J, Ng J, Fallarme-Majam V, Abot E, Goh L, Parker S, Kumar S, Hedstrom R C, Norman J, Stout R, Hoffman S L. Safety, tolerability, and lack of antibody responses after administration of a PfCSP DNA malaria vaccine via needle or needle-free jet injection, and comparison of intramuscular and combination intramuscular/intradermal routes. Hum Gene Ther. 2002 Sep. 1; 13 (13):1551-60.
16. Gallup J L, Sachs J D. The economic burden of malaria. Am J Trop Med Hyg. 2001 January-February; 64 (1-2 Suppl):85-96.
17. Graves P, Gelband H. Vaccines for preventing malaria. Cochrane Database Syst Rev. 2003; (1):CD000129.
18. Greenwood B, Alonso P. Malaria vaccine trials. Chem Immunol. 2002; 80:366-95
19. Hill A V, Reece W, Gothard P, Moorthy V, Roberts M, Flanagan K, Plebanski M, Hannan C, Hu J T, Anderson R, Degano P, Schneider J, Prieur E, Sheu E, Gilbert S C. DNA-based vaccines for malaria: a heterologous prime-boost immunisation strategy. Dev Biol (Basel). 2000; 104:171-9.
20. Le T P, Coonan K M, Hedstrom R C, Charoenvit Y, Sedegah M, Epstein J E, Kumar S, Wang R, Doolan D L, Maguire J D, Parker S E, Hobart P, Norman J, Hoffman S L. Safety, tolerability and humoral immune responses after intramuscular administration of a malaria DNA vaccine to healthy adult volunteers. Vaccine. 2000 Mar. 17; 18 (18):1893-901.
21. Moore S A, Surgey E G, Cadwgan A M. Malaria vaccines: where are we and where are we going? Lancet Infect Dis. 2002 December; 2 (12):737-43.
22. Moorthy V, Hill A V. Malaria vaccines. Br Med Bull. 2002; 62:59-72.
23. McShane H. Prime-boost immunization strategies for infectious diseases. Curr Opin Mol Ther. 2002 February; 4 (1):23-7.
24. Newman M J. Heterologous prime-boost vaccination strategies for HIV-1: augmenting cellular immune responses. Curr Opin Investig Drugs. 2002 March; 3 (3):374-8.
25. Pardoll D M. Spinning molecular immunology into successful immunotherapy. Nat Rev Immunol. 2002 April; 2 (4):227-38.
26. Richie T L, Saul A. Progress and challenges for malaria vaccines. Nature. 2002 Feb. 7; 415 (6872):694-701.
27. Rogers W O, Weiss W R, Kumar A, Aguiar J C, Tine J A, Gwadz R, Harre J G, Gowda K, Rathore D, Kumar S, Hoffman S L. Protection of rhesus macaques against lethal Plasmodium knowlesi malaria by a heterologous DNA priming and poxvirus boosting immunization regimen.
28. Infect Immun. 2002 August; 70 (8):4329-35. Rogers 2002
29. Rogers W O, Baird J K, Kumar A, Tine J A, Weiss W, Aguiar J C, Gowda K, Gwadz R, Kumar S, Gold M, Hoffman S L. Multistage multiantigen heterologous prime boost vaccine for Plasmodium knowlesi malaria provides partial protection in rhesus macaques. Infect Immun. 2001 September; 69 (9):5565-72.
30. Sedegah M, Hedstrom R, Hobart P, Hoffman S L. Protection against malaria by immunization with plasmid DNA encoding circumsporozoite protein. Proc Natl Acad Sci USA. 1994 Oct. 11; 91 (21):9866-70.
31. Sedegah M, Jones T R, Kaur M, Hedstrom R, Hobart P, Tine J A, Hoffman S L. Boosting with recombinant vaccinia increases immunogenicity and protective efficacy of malaria DNA vaccine. Proc Natl Acad Sci USA. 1998 Jun. 23; 95 (13):7648-53.
32. Stoute J A, Slaoui M, Heppner D G, Momin P, Kester K E, Desmons P, Welide B T, Garcon N, Krzych U, Marchand M. A preliminary evaluation of a recombinant circumsporozoite protein vaccine against Plasmodium falciparum malaria. RTS,S Malaria Vaccine Evaluation Group. N Engl J. Med. 1997 Jan. 9; 336 (2):86-91.
33. Stoute J A, Kester K E, Krzych U, Welide B T, Hall T, White K, Glenn G, Ockenhouse C F, Garcon N, Schwenk R, Lanar D E, Sun P, Momin P, Wirtz R A, Golenda C, Slaoui M, Wortmann G, Holland C, Dowler M, Cohen J, Ballou W R. Long-term efficacy and immune responses following immunization with the RTS,S malaria vaccine. J Infect Dis. 1998 October; 178 (4):1139-44.
34. Wang R, Doolan D L, Le T P, Hedstrom R C, Coonan K M, Charoenvit Y, Jones T R, Hobart P, Margalith M, Ng J, Weiss W R, Sedegah M, de Taisne C, Norman J A, Hoffman S L. Induction of antigen-specific cytotoxic T lymphocytes in humans by a malaria DNA vaccine. Science. 1998a Oct 16; 282 (5388):476-80.
35. Wang R, Doolan D L, Charoenvit Y, Hedstrom R C, Gardner M J, Hobart P, Tine J, Sedegah M, Fallarme V, Sacci J B Jr, Kaur M, Klinman D M, Hoffman S L, Weiss W R. Simultaneous induction of multiple antigen-specific cytotoxic T lymphocytes in nonhuman primates by immunization with a mixture of four Plasmodium falciparum DNA plasmids.
36. Infect Immun. 1998b September; 66 (9):4193-202.
37. Zavala F, Rodrigues M, Rodriguez D, Rodriguez J R, Nussenzweig R S, Esteban M. A striking property of recombinant poxviruses: efficient inducers of in vivo expansion of primed CD8(+) T cells. Virology. 2001 Feb. 15; 280 (2):155-9

Obviously, many modifications and variations of the present invention are possible in light of the above teachings. It is therefore to be understood that, within the scope of the appended claims, the invention may be practiced otherwise than as specifically described.

1. A method to enhance immunogenicity and protective immunity induced by any subunit or whole organism vaccine or combination of vaccines comprising:
a). a priming immunization preparation containing an antigen or fragment thereof, said preparation being selected from the group consisting of:
1) a recombinant virus expression system;
2) a recombinant protein antigen or a recombinant polypeptide;
3) synthetic peptide;
4) a polynucleotide vector
5) a whole organism or extract thereof; and/or
6) a combination thereof; and
b). a boosting immunization of at least one alphavirus replicon containing an antigen or fragment thereof.

2. The method of claim 1, wherein said replicon is selected from the group consisting of VEE virus, Semiliki Forest virus and Sindbis virus.

3. The method of claim 1, wherein said replicon comprises at least one Plasmodium antigen, fragment thereof, or epitope derived from said antigen, or a combination thereof.

4. The method of claim 1, wherein said replicon is selected from the group consisting of DNA replicons, RNA replicons, and replicon particles.

5. The method of claim 3, wherein said Plasmodium antigen of said replicon, fragment or epitope thereof, is selected from the group consisting of Plasmodium falciparum, Plasmodium vivax, and Plasmodium ovale, or a combination thereof.

6. The method of claim 3, wherein said Plasmodium antigen of said replicon, fragment or epitope thereof is expressed at one or more stages of the parasite life cycle, or a combination thereof.

7. The method of claim 6, wherein said life cycle stage is selected from the group consisting of preerythrocytic, erythrocytic, and transmission blocking.

8. The method of claim 2, wherein said antigen of said replicon, fragment or epitope thereof, is PyCSP

9. The method of claim 2, wherein said antigen of said replicon, fragment or epitope thereof, is PyHEP17.

10. The method of claim 2, wherein said antigen of said replicon, fragment or epitope thereof, is PfCSP.

11. The method of claim 2, wherein said antigen of said replicon fragment or epitope thereof, is PfEXP1.

12. The method of claim 2, wherein said antigen of said replicon, fragment or epitope thereof, is selected from the group consisting of PfCSP, PfExp-1, PfSSP2, PfLSA-1, PfLSA-3, PfMSP-1, PfAMA-1, PfEBA-175, PtMSP-2, PfMSP-3, PfMSP-4, PfMSP-5, PfRAP-1, PfRAP-2, or a combination thereof.

13. The method of claim 1, wherein said antigen of said replicon, fragment or epitope thereof, is obtained from an infectious disease agent.

14. The method of claim 1, wherein said virus expression system is selected from the group consisting of poxvirus, adenovirus, adenoassociated virus, and retrovirus.

15. The method of claim 14, wherein said poxvirus is selected from the group consisting of cowpox, canarypox, monkeypox, and fowlpox.

16. The method of claim 14, wherein said recombinant virus expression system comprises at least one Plasmodium antigen, fragment thereof, or epitope derived from said antigen, or combination thereof.

17. The method of claim 16, wherein said antigen of said recombinant virus expression system is a whole antigen, fragment thereof, or epitope derived from said antigen, or a combination thereof.

18. The method of claim 17, wherein said Plasmodium antigen of said recombinant virus expression system or fragment thereof is selected from the group consisting of Plasmodium falciparum, Plasmodium vivax, and Plasmodium ovale, or a combination thereof.

19. The method of claim 17, wherein said Plasmodium antigen of said recombinant virus expression system is expressed at one or more stages of the parasite life cycle, where said life cycle stage is selected from the group consisting of preerythrocytic, erythrocytic, and transmission blocking.

20. The method of claim 17, wherein said antigen of said recombinant virus expression system is obtained from an infectious disease agent.

21. The method of claim 1, wherein said recombinant protein or recombinant polypeptide comprises at least one Plasmodium antigen or fragment thereof, or epitope thereof, or epitope derived from said antigen or combination thereof.

22. The method of claim 21, wherein said Plasmodium antigen is selected from the group consisting of Plasmodium falciparum, Plasmodium vivax, and Plasmodium ovale, or a combination thereof.

23. The method of claim 21, wherein said Plasmodium antigen of said protein antigen or recombinant polypeptide or fragment thereof is expressed at one or more stages of the parasite life cycle, where said life cycle stage is selected from the group consisting of preerythrocytic, erythrocytic, and transmission blocking.

24. The method of claim 21, wherein said Plasmodium antigen of said protein antigen or recombinant polypeptide is obtained from an infectious disease agent.

25. The method of claim 1, wherein said synthetic peptide is derived from at least one Plasmodium antigen or at least one fragment thereof.

26. The method of claim 25, wherein said Plasmodium antigen or fragment thereof of said synthetic peptide is selected from the group consisting of Plasmodium falciparum, Plasmodium vivax, and Plasmodium ovale, or a combination thereof.

27. The method of claim 25, wherein said Plasmodium antigen or fragment thereof of said synthetic peptide is expressed at one or more stages of the parasite life cycle, where said life cycle stage is selected from the group consisting of preerythrocytic, erythrocytic, and transmission blocking.

28. The method of claim 25, wherein said Plasmodium antigen or fragment thereof of said synthetic peptide is obtained from an infectious disease agent.

29. The method of claim 1, wherein said polynucleotide vector comprises at least one Plasmodium antigen fragment thereof, or epitope derived from said antigen or combination thereof.

30. The method of claim 29, wherein said Plasmodium antigen of said polynucleotide vector is selected from the group consisting of Plasmodium falciparum, Plasmodium vivax, and Plasmodium ovale or a combination thereof.

31. The method of claim 29, wherein said Plasmodium antigen of said polynucleotide vector is expressed at one or more stages of the parasite life cycle, where said life cycle stage is selected from the group consisting of preerythrocytic, erythrocytic, and transmission blocking.

32. The method of claim 29, wherein said antigen of said polynucleotide vector is obtained from an infectious disease agent.

33. The method of claim 1, wherein said whole organism or extract thereof is derived from a Plasmodium species parasite.

34. The method of claim 33, wherein said organism or extract is selected from the group consisting of Plasmodium falciparum, Plasmodium vivax, and Plasmodium ovale, or a combination thereof.

35. The method of claim 33, wherein said organism or extract is expressed at one or more stages of the parasite life cycle, where said life cycle stage is selected from the group consisting of preerythrocytic, erythrocytic, and transmission blocking.

36. The method of claim 33, wherein said organism is an infectious disease agent.

37. The method of claim 1, wherein the number of doses of priming agent is 1-4 and the number of doses of boosting agent is 1-4.

38. The method of claim 1, wherein replicons are administered by routes selected from the group consisting of subcutaneous, intramuscular, intradermal, mucosal, oral, transcutaneous, and by specialized injection devices, or combinations thereof.

39. The method of claim 1, wherein recombinant viruses are administered by routes selected from the group consisting of subcutaneous, intramuscular, intradermal, mucosal, oral, transcutaneous, and by injection devices, or combinations thereof

40. The method of claim 1, wherein recombinant proteins or polypeptides are administered by routes selected from the group consisting of subcutaneous, intramuscular, intradermal, mucosal, oral, transcutaneous, and by injection devices, or combinations thereof.

41. The method of claim 1, wherein synthetic peptides are administered by routes selected from the group consisting of subcutaneous, intramuscular, intradermal, mucosal, oral, transcutaneous, and by injection devices, or combinations thereof.

42. The method of claim 1, wherein polynucleotide vectors are administered by routes selected from the group consisting of subcutaneous, intramuscular, intradermal, mucosal, oral, transcutaneous, and by specialized injection devices, or combinations thereof.

43. The method of claim 1, wherein whole organisms or extracts thereof are administered by routes selected from the group consisting of subcutaneous, intramuscular, intradermal, mucosal, oral, transcutaneous, and by injection devices, or combinations thereof.

44. The method of claim 1, wherein replicons are administered at pharmaceutically effective doses or suboptimal doses ranging between 10E3 and 10E11 replicon units

45. A method to reduce the amount of vaccine required to achieve protective immunity induced by any subunit or whole organism vaccine or combination of vaccines using a vaccination regimen comprising;
a). a priming immunization preparation containing an antigen or fragment thereof, said preparation being selected from the group consisting of:
1) a recombinant virus expression system;
2) a recombinant protein antigen or a recombinant polypeptide;
3) synthetic peptide;
4) a polynucleotide vector
5) a whole organism or extract thereof; and/or
6) a combination thereof; and
b). a boosting immunization of at least one alphavirus replicon containing an antigen or fragment thereof

46. The method of claim 45, wherein sterile protection is obtained.

47. The method of claim 45, wherein partial protection is obtained.

48. The method of claim 45, wherein a reduction in liver stage parasite burden is obtained.

49. A method to increase the duration of protective immunity induced by any subunit or whole organism vaccine or combination of vaccines using a vaccination regimen comprising;
a). a priming immunization preparation containing an antigen or fragment thereof, said preparation being selected from the group consisting of:
1) a recombinant virus expression system;
2) a recombinant protein antigen or a recombinant polypeptide;
3) a synthetic peptide;
4) a polynucleotide vector
5) a whole organism or extract thereof; and
6) a combination thereof; and
b). a boosting immunization of at least one alphavirus replicon containing an antigen or fragment thereof

50. The method of claim 49, wherein said protective immunity is maintained for 1-2 months.

51. The method of claim 49, wherein said protective immunity is maintained for 6-9 months.

52. The method of claim 49, wherein said protective immunity is maintained for 9-12 months.

53. The method of claim 49, wherein said protective immunity is maintained for 12-24 months.

54. The method of claim 49, wherein said protective immunity is maintained for 2-5 years.

55. The method of claim 49, wherein said protective immunity is maintained for 5-10 years.

56. A method to broaden the coverage in a vaccinated population of the immunogenicity and protective efficacy induced by any subunit or whole organism vaccine or combination of vaccines comprising;
a). immunizing with a priming immunization preparation containing an antigen or fragment thereof, said preparation being selected from the group consisting of:
1) a recombinant virus expression system;
2) a recombinant protein antigen or a recombinant polypeptide;
3) a synthetic peptide;
4) a polynucleotide vector
5) a whole organism or extract thereof; and/or
6) a combination thereof; and
b). immunizing subsequently with a boosting immunization preparation of at least one alphavirus replicon containing an antigen or fragment thereof.

57. The method of claim 56, wherein an immune response comprising CD8+ T cells, CD4+ T cells, or antibodies, or a combination thereof, is obtained.

58. The method of claim 56, wherein epitopes recognized by said CD8+ T cells, CD4+ T cells, antibodies or a combination thereof, said epitopes producing a response selected from the group consisting of: immunodominant, subdominant, linear or conformational.

59. The method of claim 56, wherein said immune response or protective immunity is manifested on multiple genetic backgrounds.

60. The method of claim 56, wherein the proportion of subject population responding to said vaccination with said immune response or protective immunity is increased relative to population that does not respond.
